(19) United States
(12) Patent Application Publication
(10) Pub. No.: US 2011/0319594 A1
(43) Pub. Date: Dec. 29, 2011

(54) METHOD FOR PRODUCING BIVALIRUDIN

(75) Inventors: Juncai BAI, Shanghai (CN); Ruoping ZHANG, Shanghai (CN); Yadong LIU, Shanghai (CN); Guoqing ZHANG, Shanghai (CN)

(73) Assignee: SHANGHAI AMBIOPHARM, INC., Shanghai (CN)

(21) Appl. No.: 13/158,451

(22) Filed: Jun. 12, 2011

(30) Foreign Application Priority Data
Jun. 28, 2010 (CN) 201010214547.3

(51) Int. Cl. C07K 7/08

(52) U.S. Cl. 530/326

(57) ABSTRACT

A method for producing bivalirudin using solid phase peptide synthesis by: a) condensing Fmoc-Asn(Trt)-Gly-OH with a peptide resin of Asp(OtBu)¹¹-Phe¹²-Glu(OtBu)¹³-Glu(OtBu)¹⁴-Ile¹⁵-Pro¹⁶-Glu(OtBu)¹⁷-Glu(OtBu)¹⁸-Tyr(tBu)¹⁹-Leu²⁰-Resin; b) removing Fmoc-; c) condensing Fmoc-Gly-Gly-Gly-Gly-OH with the peptide resin; d) separately condensing Pro, Arg, Pro, and Phe with the peptide resin from C-terminal to N-terminal to yield a peptide resin of Boc-D-Phe¹-Pro²-Arg(Pbf)³-Pro⁴-Gly⁵-Gly⁶-Gly⁷-Gly⁸-Asn(Trt)⁹-Gly¹⁰-Asp(OtBu)¹¹-Phe¹²-Glu(OtBu)¹³-Glu(OtBu)¹⁴-Ile¹⁵-Pro¹⁶-Glu(OtBu)¹⁷-Glu(OtBu)¹⁸-Tyr(tBu)¹⁹-Leu²⁰-Resin; and e) in the presence of a cleavage agent, separating a peptide from the peptide resin to yield bivalirudin represented by Formula VI. The method is low in cost and the resultant bivalirudin has high purity.

### CROSS-REFERENCE TO RELATED APPLICATIONS

Pursuant to 35 U.S.C. §119 and the Paris Convention Treaty, this application claims the benefit of Chinese Patent Application No. 201010214547.3 filed on Jun. 28, 2010, the contents of which are incorporated herein by reference.

### BACKGROUND OF THE INVENTION

1. Field of the Invention

The invention relates to a method for producing a peptide, and more particularly to a method for producing bivalirudin using solid phase peptide synthesis.

2. Description of the Related Art

Solid phase peptide synthesis, a breakthrough for producing peptide, was invented by R. Bruce Merrifield. Solid phase peptide synthesis involves: linking a first amino acid whose amino group is protected by a protecting group to a solid phase support, removing the protecting group with a de-protective agent, activating a carboxyl of a protected second amino acid with N,N′-dicyclohexyl carbodiimide (DCC), and reacting the first amino acid with the second amino acid to yield a protected dipeptide at the solid phase support. When the above steps are repeated, the peptide chain grows from C-terminal to N-terminal. After the required chain length is obtained, the protecting group is removed, and the ester bond between the peptide chain and the solid phase carrier is hydrolyzed with the strong acid HF. In this way, a peptide is obtained. The synthesis technique is actually a process of adding amino acid repetitively, and the synthesis order is from the C-terminal (carboxyl terminal) to the N-terminal (amino terminal).

Thrombin inhibitors are considered as a promising anti-thrombosis drug. Bivalirudin, an anticoagulant peptide, is a bivalent hirudin (hirulog). Hirudin is a peptide therapeutically effective for inhibiting thrombin and extracted from a blood-sucking leech, i.e., Hirudo medicinalis, with 20 amino acids. Bivalirudin is a direct thrombin inhibitor widely applied in clinic recently and was approved for marketing in 2000, USA, and the active ingredient thereof is hirudin derivatives. The anticoagulant effect of bivalirudin is reversible and short-lived. Bivalirudin is an ideal substitute of antagonists of normal hepatic cord and platelet glycoprotein IIb/IIa for percutaneous coronary intervention.

US20070093423A discloses a method for producing bivalirudin using solid phase peptide synthesis. Actually, it is a combination of solid phase and liquid phase synthesis method which is very difficult for practice. In addition, the cleavage agent used therein includes acids, ethanedithiol, etc. Thus, the method has high cost, and the resultant product has many impurities.

US20090062511A discloses a method for producing bivalirudin using solid phase peptide synthesis, which involves complicated operation and results in impurities.

Thus, it is very urgent to design a method for producing bivalirudin using solid phase peptide synthesis that has low cost and by which the resultant bivalirudin has high purity, particularly the glycine-deletion and glycine addition closely eluted with the main peak of HPLC can be reduced after the preparative HPLC purification to meet the pharmaceutical requirements.

### SUMMARY OF THE INVENTION

In view of the above-described problems, it is one objective of the invention to provide a method for producing bivalirudin using solid phase peptide synthesis.

To achieve the above objective, in accordance with one embodiment of the invention, there is provided a method for producing bivalirudin using solid phase peptide synthesis, the method comprising

a) in the presence of a condensing agent, condensing Fmoc-Asn(Trt)-Gly-OH with a peptide resin represented by Formula I (SEQ ID NO. 1);

Asp(OtBu)¹¹-Phe¹²-Glu(OtBu)¹³-Glu(OtBu)¹⁴-Ile¹⁵-Pro¹⁶-Glu(OtBu)¹⁷-Glu(OtBu)¹⁸-Tyr(tBu)¹⁹-Leu²⁰-Resin (I)

to yield a peptide resin represented by Formula II (SEQ ID NO. 2);

Fmoc-Asn(Trt)⁹-Gly¹⁰-Asp(OtBu)¹¹-Phe¹²-Glu(OtBu)¹³-Glu(OtBu)¹⁴-Ile¹⁵-Pro¹⁶-Glu(OtBu)¹⁷-Glu(OtBu)¹⁸-Tyr(tBu)¹⁹-Leu²⁰-Resin (II)

b) mixing the peptide resin represented by Formula II with a de-protective agent to remove Fmoc- and yield a peptide resin represented by Formula III (SEQ ID NO. 3);

Asn(Trt)⁹-Gly¹⁰-Asp(OtBu)¹¹-Phe¹²-Glu(OtBu)¹³-Glu(OtBu)¹⁴-Ile¹⁵-Pro¹⁶-Glu(OtBu)¹⁷-Glu(OtBu)¹⁸-Tyr(tBu)¹⁹-Leu²⁰-Resin (III)

c) in the presence of the condensing agent, condensing Fmoc-Gly-Gly-Gly-Gly-OH with the peptide resin represented by Formula III to yield a peptide resin represented by Formula IV (SEQ ID NO. 4);

Fmoc-Gly⁵-Gly⁶-Gly⁷-Gly⁸-Asn(Trt)⁹-Gly¹⁰-Asp(OtBu)¹¹-Phe¹²-Glu(OtBu)¹³-Glu(OtBu)¹⁴-Ile¹⁵-Pro¹⁶-Glu(OtBu)¹⁷-Glu(OtBu)¹⁸-Tyr(tBu)19-Leu²⁰-Resin (IV)

d) separately condensing Pro, Arg, Pro, and Phe with the peptide resin represented by Formula IV from C-terminal to N-terminal to yield a peptide resin represented by Formula V (SEQ ID NO. 5),

Boc-D-Phe¹-Pro²-Arg(Pbf)³-Pro⁴-Gly⁵-Gly⁶-Gly⁷-Gly⁸-Asn(Trt)⁹-Gly¹⁰-Asp(OtBu)¹¹-Phe¹²-Glu(OtBu)¹³-Glu(OtBu)¹⁴-Ile¹⁵-Pro¹⁶-Glu(OtBu)¹⁷-Glu(OtBu)¹⁸-Tyr(tBu)¹⁹-Leu²⁰-Resin (V)

and

e) in the presence of a cleavage agent, separating a peptide from the peptide resin represented by Formula V to yield bivalirudin represented by Formula VI (SEQ ID NO. 6).

D-Phe-Pro-Arg-Pro-Gly-Gly-Gly-Gly-Asn-Gly-Asp-Phe-Glu-Glu-Ile-Pro-Glu-Glu-Tyr-Leu (VI)

In a class of this embodiment, Fmoc-Asn(Trt)-Gly-OH is synthesized as follows: a) mixing Z-Asn(Trt)-OH with H-Gly-OBzl.TosOH so that a liquid phase peptide condensation reaction happens between the two to yield Z-Asn(Trt)-Gly-OBzl; b) reducing Z-Asn(Trt)-Gly-OBzl with hydrogen to yield H-Asn(Trt)-Gly-OH; and c) mixing H-Asn(Trt)-Gly-OH with Fmoc to yield Fmoc-Asn(Trt)-Gly-OH.

In a class of this embodiment, Fmoc-Gly-Gly-Gly-Gly-OH is synthesized as follows: a) mixing H-Gly-Gly-OBzl with Z-Gly-Gly-OH so that a liquid phase peptide condensation reaction happens between the two to yield Z-Gly-Gly-Gly-Gly-OBzl; b) reducing Z-Gly-Gly-Gly-Gly-OBzl with hydrogen to yield H-Gly-Gly-Gly-Gly-OH; and c) mixing H-Gly-Gly-Gly-Gly-OH with Fmoc to yield Fmoc-Gly-Gly-Gly-Gly-OH.

In a class of this embodiment, H-Gly-Gly-OBzl is synthesized by condensing Boc-Gly-OH and H-Gly-OBzl using liquid phase peptide condensation and then removing protecting groups of the condensate.

In a class of this embodiment, Z-Gly-Gly-OH is synthesized by condensing Z-Gly-OH and H-Gly-OMe using liquid phase peptide condensation and then reducing the condensate.

In a class of this embodiment, based on its total volume, the de-protective agent comprises between 3 and 20% of piperidine and between 0.5 and 10% of bicyclic amidine (DBU).

In a class of this embodiment, the de-protective agent further comprises between 0 and 20% of 1-hydroxy benzotriazole (HOBt), between 0 and 8% of 3-hydroxy-1,2,3-benzo triazine-4(3H)-one (HOOBt), or a mixture thereof.

In a class of this embodiment, based on its total volume, the de-protective agent comprises between 5 and 15% of piperidine and between 1 and 7% of bicyclic amidine (DBU).

In a class of this embodiment, the de-protective agent further comprises between 0.5 and 10% of 1-hydroxy benzotriazole (HOBt), between 2 and 5% of 3-hydroxy-1,2,3-benzo triazine-4(3H)-one (HOOBt), or a mixture thereof.

In a class of this embodiment, in the step d), upon condensing Arg, Fmoc-Arg(Pbf)-OH, pentafluorophenol, and the condensing agent are mixed so as to prompt the condensation of Fmoc-Arg(Pbf)-OH with the peptide bound to the resin.

In a class of this embodiment, the condensing agent is N,N′-diisopropyl carbodiimide (DIC), O-(7-aza-benzotriazole-1-yl)-N,N,N′,N′-tetramethyl uronium hexafluoro phosphate (HATU), O-(benzotriazole-1-yl)-N,N,N,N-4-methyl-uronium tetrafluoroborate (TBTU)/N-methyl morpholine (NMM) or diisopropyl ethylamine (DIEA), O-(7-benzotriazole-1-yl)-N,N,N′,N′-tetramethyl uronium hexafluoro phosphate (HBTU)/N-methyl morpholine (NMM) or diisopropyl ethylamine (DIEA), (benzo triazol-1-yl-O)tripyrrolidine phosphonium hexafluorophosphate (PyBOP), 1-hydroxy benzotriazole (HOBt), or a mixture thereof.

In a class of this embodiment, the peptide condensation process is monitored using ninhydrin colorimetric method (Kaiser).

In a class of this embodiment, the peptide condensation after introduction of proline (Pro) is monitored using Chloranil and Kaiser test method.

In a class of this embodiment, the cleavage agent comprises trifluoroacetic acid (TFA), triisopropyl silane (TIS), and water, with a volume ratio thereof 95-60: 5-10: 5-30.

Thus, the invention provides a method for producing bivalirudin using solid phase peptide synthesis that has low cost and by which the resultant bivalirudin has high purity, particularly the glycine-deletion and glycine-addition closely eluted with the main peak of HPLC can be reduced to less than 0.6% and 0.2%, respectively, after the preparative HPLC purification to meet the pharmaceutical impurity requirements.

Advantages of the Invention are Summarized Below:
1) Low cost: compared with conventional methods for producing bivalirudin, the method reduces cost by about 50%;
2) Low impurities and high purity: the glycine-deletion and glycine-addition impurities closely eluted with the main peak can be controlled less than 0.6% and 0.2%, respectively, after the preparative HPLC purification to meet the pharmaceutical requirements;
3) Low risk factor: methyl tert-butyl ether (MTBE) instead of ether is used in the invention, which improves the safety of production. Ether is an extremely flammable chemical with flash point of −45° C., and boiling point of 34.6° C. Methyl tert-butyl ether has a flash point of −28° C. and a boiling point of 55.3° C.; and
4) environment-friendly: the method is a solid phase synthesis process, almost no water involved in, and the organic solvents for washing can be recycled and thereby waste is very little.

### DETAILED DESCRIPTION OF THE EMBODIMENTS

The invention is described hereinbelow with reference to accompanying drawings, in which:

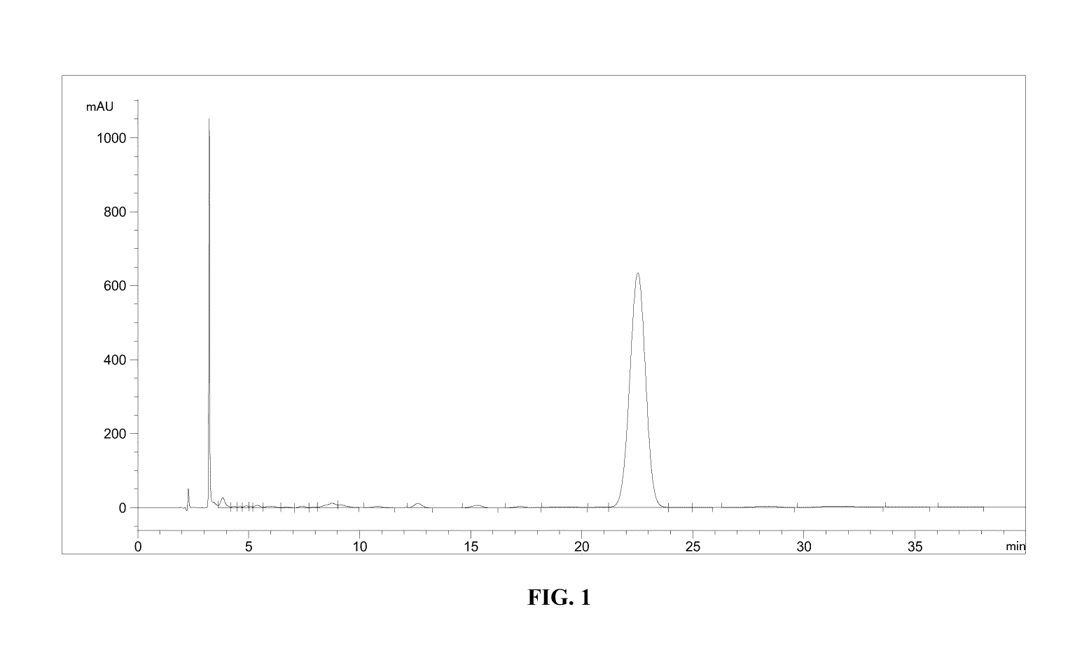

FIG. 1 is an HPLC chromatogram of bivalirudin according to one embodiment of the invention;

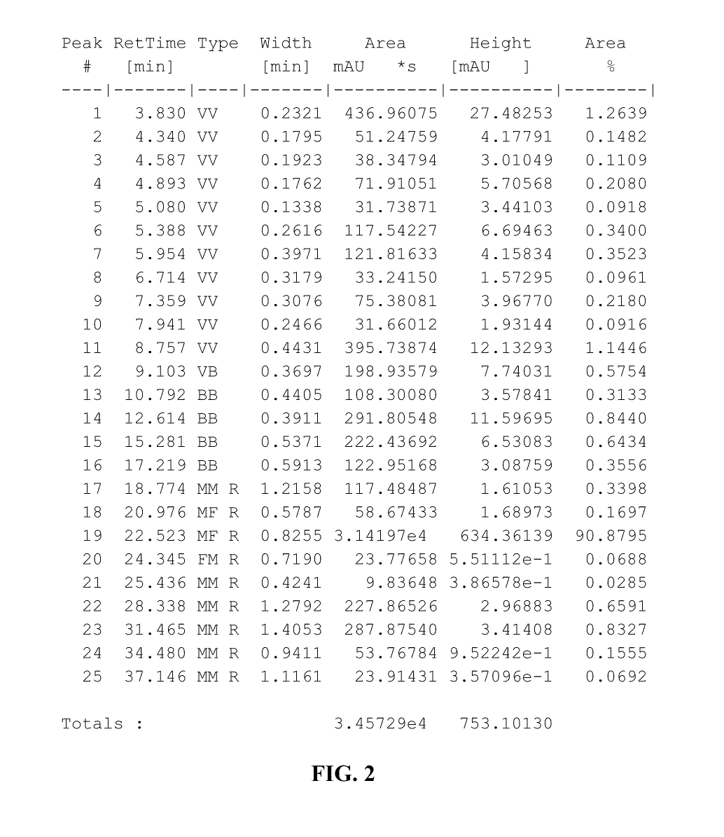

FIG. 2 is a data sheet of the HPLC chromatogram of bivalirudin of FIG. 1;

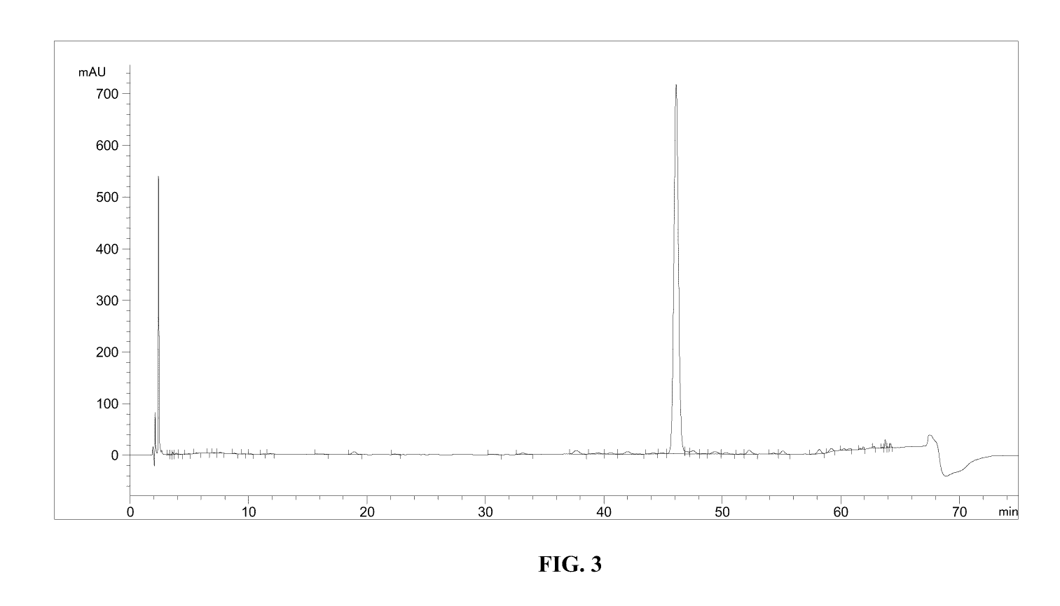

FIG. 3 is an HPLC chromatogram of bivalirudin according to another embodiment of the invention; and

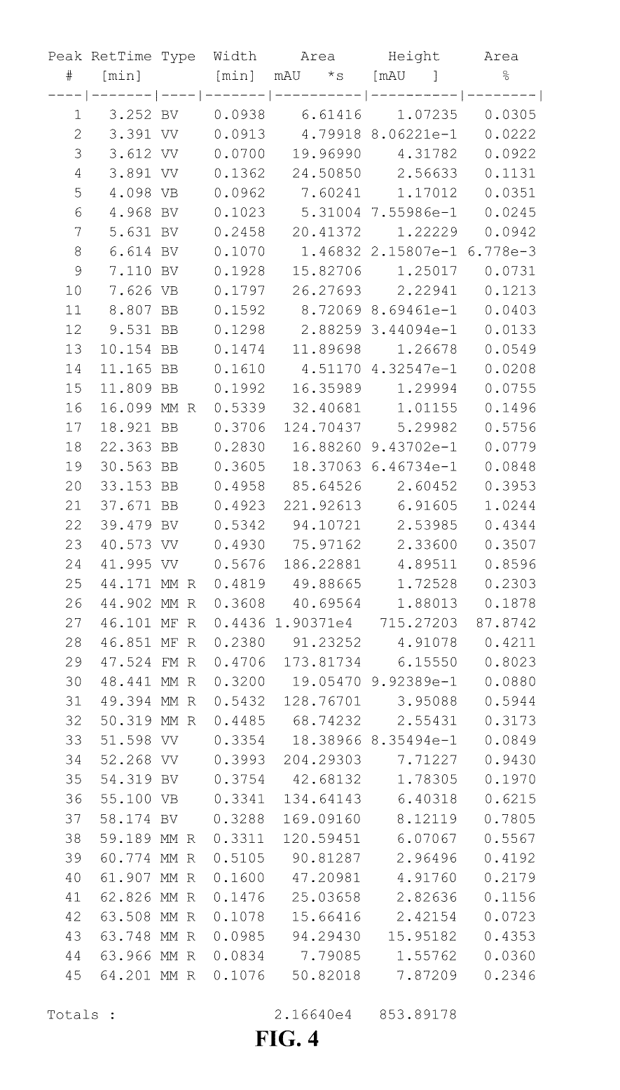

FIG. 4 is a data sheet of the HPLC chromatogram of bivalirudin of FIG. 3.

### DETAILED DESCRIPTION OF THE EMBODIMENTS

Studies show that condensing Asn(Trt)-Gly with Gly-Gly-Gly-Gly by using solid phase peptide synthesis to produce bivalirudin simplifies the purification process and can effectively remove impurity peaks (that is, the glycine-deletion and glycine-addition closely eluted with the main peak of HPLC), thereby improving the purification efficiency.

In embodiments of the invention, a de-protective agent comprising DMF, piperidine, DBU, HOBt, or HOOBT, particularly for the structure of -Asn-Gly-, a de-protective agent comprising piperidine, DBU, HOBt, HOOBT, or a mixture thereof is highly effective.

In addition, studies show that during producing bivalirudin using solid phase peptide synthesis, upon condensation, a condensing agent comprising HOBt/DIC or TBTU/NMM can be added and the whole condensation process can be monitored. For steps of condensing Arginine (Arg), pentafluorophenol is required so as to reduce the production cost and reduce the Arginine deletion impurity.

The meaning of the abbreviations of the invention is listed as follows:

| | |
|---|---|
| Fmoc | 9-fluorenylmethoxycarbonyl |
| Z | Carbobenzoxy group |
| Fmoc-Osu | 9-fluorenyl-methoxyl-n-succinimide |
| Boc | Butoxycarbonyl |
| DMF | N,N-dimethylformamide |
| KSCN | Potassium thiocyanate |
| DBU | 1,8-diazabicyclo(5.4.0)undec-7-ene |
| HOBt | 1-hydroxy benzotriazole |
| DIC | N,N'-diisopropyl c + arbodiimide |
| TBTU | O-(benzotriazole-1-yl)-N,N,N,N-4-methyl-uronium |
| | tetrafluoroborate |
| NMM | N-methyl morpholine |
| HBTU | O-(7-benzotriazole-1-y1)-N,N,N',N'-tetramethyl |
| | uronium hexafluoro phosphate |
| DIEA | Diisopropyl ethylamine |
| Pbf | 2,2,4,6,7-5-pentamethyl-benzofuran-5-sulfonyl |
| Opfp | Pentafluorophenyl ester |
| TFA | Trifluoroacetic acid |
| TIS | Triisopropyl silane |
| MTBE | Methyl tert-butyl ether |
| HOOBT | 3-hydroxy-1,2,3-benzo triazine-4(3H)-one |
| HATU | O-(7-aza-benzotriazole-1-yl)-N,N,N',N'-tetramethyl |
| | uranium hexafluoro phosphate |
| PyBOP | (benzo triazol-1-yl-O)tripyrrolidine phosphonium |
| | hexafluorophosphate |
| EtOAc | Ethyl acetate |
| OBzl | Benzyl |
| TosOH | Tosylate |
| EDC | 1-ethyl-(3-dimethyl aminopropyl)carbodiimide |
| tBu | Tert-butyl, —C(CH₃)₃ |
| OtBu | —O—C(CH₃)₃ |

In embodiments of the invention, “solid phase synthesis” or “solid phase peptide phase” is well-known to one of ordinary skill in the art, comprising but not limited to the following steps: a) covalently binding a first amino acid whose amino-group is blocked to a solid phase carrier; b) in the presence of a de-protective agent, removing the protecting group of the amino-group; c) activating the carboxyl of a second amino acid with dicyclohexylcarbodiimide (DCC) whose amino-group is blocked and contacting the second amino acid with the first amino acid bound to the solid phase carrier so that a dipeptide whose amino-group is blocked is obtained; c) repeating the peptide bond formation steps and thus the peptide chain is extended from C-terminal to N-terminal; and d) removing the protecting group of the amino-group and separating the peptide chain from the solid phase carrier with a cleavage agent to yield a peptide.

In embodiments of the invention, the de-protective agent is a chemical agent which can remove a protecting group of amino group. The protecting group of amino group is well-known to those of ordinary skill in the art and includes but is not limited to Fmoc and Boc. Particularly, based on its total volume, the de-protective agent comprises between 3 and 20% of piperidine and between 0.5 and 10% of bicyclic amidine (DBU). More particularly, the de-protective agent further comprises between 0 and 20% of 1-hydroxy benzotriazole (HOBt), between 0 and 10% of 3-hydroxy-1,2,3-benzo triazine-4(3H)-one (HOOBt), or a mixture thereof.

In embodiments of the invention, the condensing agent is a chemical agent which can prompt the formation of a peptide bond between an amino group of an amino acid and a carboxyl of another amino acid. The condensing agent is well-known to those of ordinary skill in the art and includes but is not limited to carbodiimide, ByPOB, HATU, and TBTU.

In embodiments of the invention, the cleavage agent is a chemical agent which can separate a peptide bound to a resin from the resin. The cleavage agent is well-known to those of ordinary skill in the art and includes but is not limited to a weak acid solution comprising TFA and HCl solution.

In embodiments of the invention, a method for producing bivalirudin using solid phase peptide synthesis comprises
a) loading Fmoc-Leu-OH to a resin; the resin is well-known to those of ordinary skill in the art and particularly a Wang resin, and more particularly a Wang resin having a substitution rate of 0.6-1.4 mmol/g;
b) removing Fmoc- with a de-protective agent, i.e., washing the Fmoc-Leu-resin of step a) with the de-protective agent so as to remove Fmoc-;
c) condensing Fmoc-Tyr(tBu)-OH with the leucine (Leu) bound to the resin to yield a Fmoc-Tyr(tBu)-Leu-resin;
d) removing the Fmoc- with the de-protective agent;
e) repeating the above steps for peptide bond formation so that the peptide is extended from C-terminal to N-terminal until a Fmoc-Asp(OtBu)¹¹-Phe¹²-Glu(OtBu)¹³-Glu(OtBu)¹⁴-Ile¹⁵-Pro¹⁶-Glu(OtBu)¹⁷-Glu(OtBu)¹⁸-Tyr(tBu)¹⁹-Leu²⁰-resin is produced (OtBu/tBu is a protecting group and removed in the end);
f) removing the Fmoc- with the de-protective agent;
g) condensing Fmoc-Asn(Trt)-Gly-OH with the peptide bound to the resin to yield a Fmoc-Asn(Trt)⁹-Gly¹⁰-Asp(OtBu)¹¹-Phe¹²-Glu(OtBu)¹³-Glu(OtBu)¹⁴-Ile¹⁵-Pro¹⁶-Glu(OtBu)¹⁷-Glu(OtBu)¹⁸-Tyr(tBu)¹⁹-Leu²⁰-resin;
h) removing the Fmoc- with the de-protective agent;
i) condensing Fmoc-Gly-Gly-Gly-Gly-OH with the peptide bound to the resin to yield a Fmoc-Gly⁵-Gly⁶-Gly⁷-Gly⁸-Asn(Trt)⁹-Gly¹⁰-Asp(OtBu)¹¹-Phe¹²-Glu(OtBu)¹³-Glu(OtBu)¹⁴-Ile¹⁵-Pro¹⁶-Glu(OtBu)¹⁷-Glu(OtBu)¹⁸-Tyr(tBu)¹⁹-Leu²⁰-resin;
j) removing the Fmoc- with the de-protective agent;
k) repeating the above steps for peptide bond formation so that the peptide is extended from C-terminal to N-terminal until a Boc-D-Phe¹-Pro²-Arg(Pbf)³-Pro⁴-Gly⁵-Gly⁶-Gly⁷-Gly⁸-Asn(Trt)⁹-Gly¹⁰-Asp(OtBu)¹¹-Phe¹²-Glu(OtBu)¹³-Glu(OtBu)¹⁴-Ile¹⁵-Pro¹⁶-Glu(OtBu)¹⁷-Glu(OtBu)¹⁸-Tyr(tBu)¹⁹-Leu²⁰-resin; and
l) in the presence of a cleavage agent, separating the peptide represented by Formula VI from the resin to yield bivalirudin, the cleavage agent comprising TFA, TIS, and water.

Preferably, in step a), 1.0-3.0 resin equivalent of Fmoc-Leu-OH is reacted with a Wang resin.

Preferably, in steps c), e), g), i), and/or k), 1.5-4.5 resin equivalent of Fmoc-amino acid and 1.5-3.0 resin equivalent of HOBt are dissolved with DMF(1 mL/g resin); the mixture is added to the resin, and then 2.0-6.0 resin equivalent of DIC or TBTU is added, and allowed to react for 90 min. The resultant solution is diluted with DMF at 10° C. to a volume (4 mL/g resin) and then allowed for reaction for 6 hrs.

Preferably, in step k), the condensation of Fmoc-Arg(Pbf)-OH is as follows: 1.5-6.0 equivalents of Fmoc-Arg(Pbf)-OH and pentafluorophenol are dissolved with DMF (3 mL/g resin), and then 1.5-6.0 equivalents of a condensing agent such as DIC, HATU, TBTU, or PyBOP are added and stirred for 90 min. The resultant Fmoc-Arg(Pbf)-OPfp/DMF solution is added to the resin and stirred for 12-36 hrs.

In embodiments of the invention, the condensation reactions are monitored using ninhydrin colorimetric method (Kaiser). Particularly, the peptide condensation after the introduction of proline (Pro) (i.e., the condensation of the first amino acid exactly after the introduction of Pro, for example, 1#Boc-D-Phe-OH, 3#Fmoc-Arg(pbe-OH, and 15#Fmoc-Ile-OH) is monitored using Chloranil and Kaiser test method.

The ninhydrin colorimetric method (Kaiser) and Chloranil and Kaiser test method is recited in the literatures below. VIRENDER K. SARIN, et al. “Quantitative Monitoring of Solid-Phase Peptide Synthesis by the Ninhydrin Reaction” ANALYTICAL BIOCHEMISTRY 117, 147-157 (1981); E. KAISER, et al. “Color Test for Detection of Free Terminal Amino Groups in the Solid-Phase Synthesis of Peptides” SHORT COMMUNICATIONS 595-598 (Received Oct. 28, 1969); and THORKILD CHRISTENSEN “A Qualitative Test for Monitoring Coupling Completeness in Solid Phase Peptide Synthesis Using Chloranil” Acta Chemica Scandinavica B 33 (1979) 763-766.

In embodiments of the invention, the obtained crude bivalirudin has a yield of 90-125% and purity of 80-91%. Preferably, the obtained peptide represented by Formula VI is mixed with MTBE or ether to yield a peptide precipitate. More preferably, the MTBE or ether is cooled to −10 to 0° C. by an ice-water bath or a refrigerant known to those of ordinary skill in the art, and the precipitate is washed with another ether and separated by filtration or centrifugation. The purity of the resultant bivalirudin can reach 80% or more.

The above mentioned technical features can be combined freely upon implementation.

For further illustrating the invention, experiments detailing a method for producing bivalirudin using solid phase peptide synthesis are described below. It should be noted that the following examples are intended to describe and not to limit the invention.

Unless otherwise specified, the experiments in Examples are carried out at normal conditions or in accordance with the conditions recommended by the manufacturer, and all percentage, ratio, or proportion is calculated by weight.

The volume percentage of weight of the invention is well-known to those of ordinary skill in the art, e.g., the weight of solute dissolved in 100 mL of solution.

Unless otherwise specified, the meaning of scientific terms in the invention is the same as that known to those of ordinary skill in the art. Methods or materials similar to or equal to those of the invention are practical.

### EXAMPLE 1

### Preparation of Bivalirudin I

A. Preparation of Fmoc-Gly-Gly-Gly-Gly-OH Using Liquid Phase Synthesis Method

The reaction formula for the preparation of Fmoc-Gly-Gly-Gly-Gly-OH is summarized as below:

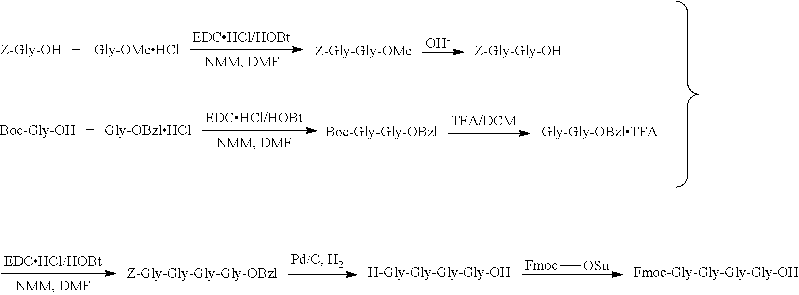

1. Preparation of Z-Gly-Gly-OMe

104.55 g of Z-Gly-OH and 69.05 g of H-Gly-OMe.HCl were dissolved in 600 mL of DMF. The solution was cooled to 0° C. using a cold bath and then 74.32 g of HOBt and 105.44 g of EDC.HCl were added. The pH value of the solution was adjusted to 8 with NMM and then the cold bath was removed so that the chemical reactions took place at room temperature. The reactions were monitored using TLC (Thin Layer Chromatography). After the reaction, the solution was diluted with EtOAc (600 mL), washed with 5% H₃PO₄ (600 mL), and extracted with 300 mL of EtOAc. The organic phase was combined and washed separately with 5% H₃PO₄ once, saturated salt water once, saturated NaHCO₃ solution thrice, and saturated salt water once, and dried over anhydrous Na₂SO₄, filtered, and concentrated to yield a white solid (Z-Gly-Gly-OMe).

2. Preparation of Z-Gly-Gly-OH

280.34 g of Z-Gly-Gly-OMe was dissolved in 200 mL of THF and the resulting solution was cooled to 0° C. 1N NaOH (150 mL) was dripped to the solution and the temperature therein was increased to 1° C., with stirring. The reactions were monitored using TLC (Thin Layer Chromatography). After the reaction, the solution was extracted with EtOAc (200 mL) thrice. The organic phase was removed and the pH value of the water phase was adjusted to 3 with 6N HCl. A solid precipitate was produced. The precipitate was filtered, collected, washed with water thrice, and dried to yield a white solid (Z-Gly-Gly-OH).

3. Preparation of Boc-Gly-Gly-OBzl

19.3 g of Boc-Gly-OH, 44.5 g of H-Gly-OBzl.TosOH, and 17.84 g of HOBt were dissolved in 500 mL of DMF. The solution was cooled to 0° C. using a cold bath and then 25.3 g of EDC.HCl were added. The pH value of the solution was adjusted to 8 with NMM and then the cold bath was removed so that the chemical reactions took place at room temperature (20° C.) overnight. After the reaction, the solution was diluted with EtOAc (EA), washed with 5% H₃PO₄, and extracted with EA. The organic phase was combined and washed separately with 5% H₃PO₄ once, saturated salt water once, saturated NaHCO₃ solution thrice, and saturated salt water once, and dried over anhydrous Na₂SO₄, filtered, and concentrated to yield a white solid (Boc-Gly-Gly-OBzl).

4. Preparation of H-Gly-Gly-OBzl.TFA

29.02 g of Boc-Gly-Gly-OBzl was dissolved in 150 mL of DCM, and at 0° C., 50 mL of TFA was added. The solution was stirred for reaction and in the end the solvent was removed to yield an oily product (H-Gly-Gly-OBzl.TFA).

5. Preparation of Z-Gly-Gly-Gly-Gly-OBzl

36 g of H-Gly-Gly-OBzl.TFA was dissolved in 500 mL of DMF, and at 0° C. NMM was added to adjust the pH value of the solution to 7. To the solution, Z-Gly-Gly-OH (22.64 g), HOBt (12.16 g), and EDC.HCl (17.25 g) were further added, and the pH value thereof was adjusted with NMM to 8. The cold bath was removed so that the chemical reactions took place at room temperature. After the reaction, a solid precipitate was produced. The precipitate was filtered, collected, washed separately with 5% H₃PO₄ and saturated NaHCO₃ solution, and dried to yield a solid (Z-Gly-Gly-Gly-Gly-OBzl).

6. Preparation of H-Gly-Gly-Gly-Gly-OH

38.5 g of Z-Gly-Gly-Gly-Gly-OBzl was dissolved in 1 L of DMF at 40° C., Pd/C (10%, 10 g) added. The solution was carried out with a hydrogenation reaction. Subsequently, the solution was filtered and the filter residue collected. The residue was stirred with 200 mL of 1N NaHCO₃. The resulting mixture was filtered and the filtrate was collected for next step.

7. Preparation of Fmoc-Gly-Gly-Gly-Gly-OH

The filtrate collected from the step 6) was cooled using a cold bath and THF solution containing Fmoc-OSu was added. The cold bath was removed so that the chemical reactions took place at room temperature. 1N HCl was added to adjust the pH value to between 2 and 3. A precipitate was filtered, collected, washed with water and EA, and dried to yield a solid.

The solid was dissolved in DMF at 70° C. The solution was added to a mixture comprising water and saturated salt water, each of which has a volume four times as much as that of the solution. A precipitate was produced, which was washed with water and dried to yield a product (Fmoc-Gly-Gly-Gly-Gly-OH, with a purity of 98.54%).

### B. Preparation of D-Phe-Pro-Arg-Pro-Gly-Gly-Gly-Gly-Asn-Gly-Asp-Phe-Glu-Glu-Ile-Pro-Glu-Glu-Tyr-Leu Using Solid Phase Synthesis Method

Loading: An amino acid resin was put into a solid-phase synthesis reactor. Each gram of the resin was swollen with 10-15 mL of DMF for 2-3 hrs.

Bonding of Fmoc-Leu: 2.0 molar equivalents of Fmoc-Leu-OH was activated with 2,6-dichlorobenzoylchloride and pyridine and reacted with Wang resin (having a substitution rate of 0.6-1.4 mmol/g) in a DMF solution.

Removing Fmoc: Another DMF solution comprising 15% of piperidine/5% of DBU was added and allowed to react for 30 min so as to remove Fmoc. The resultant resin was washed once with DMF, thrice with methanol, and thrice with DMF, respectively.

Condensing Fmoc-Gly-Gly-Gly-Gly-OH: 3.0 resin equivalent of Fmoc-Gly-Gly-Gly-Gly-OH and 3.0 resin equivalent of HOBt were dissolved with DMF (3 mL/g resin); the mixture was added to the resin, and then 3.0 resin equivalent of DIC was added. The whole process was monitored by ninhydrin colorimetric method (Kaiser). The resin was washed with methanol and DMF.

Condensing Fmoc-amino acid (the last was Boc-D-Phe-OH): 3.0 resin equivalent of Boc-D-Phe-OH (the No. 1 amino acid) and 3.0 resin equivalent of HOBt were dissolved with DMF (3 mL/g resin); the mixture was added to the resin, and then 3.0 resin equivalent of DIC was added.

Condensing Fmoc-Arg(Pbf)-OH: 3.0 equivalents of Fmoc-Arg(Pbf)-OH and pentafluorophenol were dissolved with DMF (3 mL/g resin), and then 3.0 equivalents of DIC were added and stirred for 45 min. The resultant Fmoc-Arg(Pbf)-OPfp/DMF solution was added to the resin and stirred for 3 hrs. If the condensation reaction was not complete in 21-24 hrs, repeat the above steps.

Monitoring the condensation: The whole process was monitored by ninhydrin colorimetric method (Kaiser). The peptide condensation after the introduction of proline (Pro) was monitored using Chloranil and Kaiser test method.

Washing: After all required amino acids were condensed, the resin was washed with methanol and DMF and dried to a constant weight, and a yield thereof was calculated according to its weight gain.

Preparation of a cleavage agent: TFA, TIS, and water with a volume ratio of 95:2.5:2.5 (±10%) were mixed in a vessel to yield a cleavage agent.

Cleavage: The peptide resin was slowly added to a cooled cleavage agent. The mixture was held at 0-15° C. and then increases to 18-28° C., and stirred for 2-3 hrs. The mixture was filtered and the resin washed with TFA (0.5-10. mL TFA/g resin). The resultant filtrate was collected.

Precipitating: To MTBE cooled to −8° C., the filtrate was added and a peptide precipitate was produced. The precipitate stood for 60-80 min at 0-10° C. and was separated using centrifugation. The collected precipitate was washed with methyl tert-butyl ether and separated again using centrifugation.

Drying: The solid peptide was transferred to a vessel and dried to yield bivalirudin I.

Analysis showed that the impurity Bivalirudin−Gly was 0.17% in content, and the impurity Bivalirudin+Gly was 0.07% in content.

### EXAMPLE 2

### Preparation of Bivalirudin II

### A. Preparation of Fmoc-Gly-Gly-Gly-Gly-OH Using Liquid Phase Synthesis Method

The reaction formula for the preparation of Fmoc-Gly-Gly-Gly-Gly-OH is summarized as below:

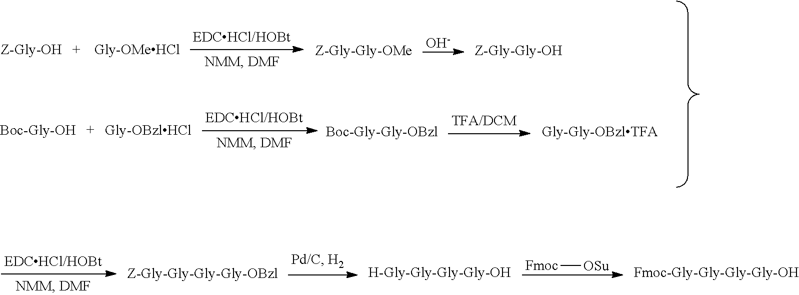

1. Preparation of Z-Gly-Gly-OMe

104.55 g of Z-Gly-OH and 69.05 g of H-Gly-OMe.HCl were dissolved in 600 mL of DMF. The solution was cooled to 0° C. using a cold bath and then 74.32 g of HOBt and 105.44 g of EDC.HCl were added. The pH value of the solution was adjusted to 8 with NMM and then the cold bath was removed so that the chemical reactions took place at room temperature. The reactions were monitored using TLC (Thin Layer Chromatography). After the reaction, the solution was diluted with EtOAc (600 mL), washed with 5% H₃PO₄ (600 mL), and extracted with 300 mL of EtOAc. The organic phase was combined and washed separately with 5% H₃PO₄ once, saturated salt water once, saturated NaHCO₃ solution thrice, and saturated salt water once, and dried over anhydrous Na₂SO₄, filtered, and concentrated to yield a white solid (Z-Gly-Gly-OMe).

2. Preparation of Z-Gly-Gly-OH

280.34 g of Z-Gly-Gly-OMe was dissolved in 200 mL of THF and the resulting solution was cooled to 0° C. 1N NaOH (150 mL) was dripped to the solution and the temperature therein was increased to 1° C., with stirring. The reactions were monitored using TLC (Thin Layer Chromatography). After the reaction, the solution was extracted with EtOAc (200 mL) thrice. The organic phase was removed and the pH value of the water phase was adjusted to 3 with 6N HCl. A solid precipitate was produced. The precipitate was filtered, collected, washed with water thrice, and dried to yield a white solid (Z-Gly-Gly-OH).

3. Preparation of Boc-Gly-Gly-OBzl

19.3 g of Boc-Gly-OH, 44.5 g of H-Gly-OBzl.TosOH, and 17.84 g of HOBt were dissolved in 500 mL of DMF. The solution was cooled to 0° C. using a cold bath and then 25.3 g of EDC.HCl were added. The pH value of the solution was adjusted to 8 with NMM and then the cold bath was removed so that the chemical reactions took place at room temperature (20° C.) overnight. After the reaction, the solution was diluted with EtOAc, washed with 5% H₃PO₄, and extracted with EA. The organic phase was combined and washed separately with 5% H₃PO₄ once, saturated salt water once, saturated NaHCO₃ solution thrice, and saturated salt water once, and dried over anhydrous Na₂SO₄, filtered, and concentrated to yield a white solid (Boc-Gly-Gly-OBzl).

4. Preparation of H-Gly-Gly-OBzl.TFA

29.02 g of Boc-Gly-Gly-OBzl was dissolved in 150 mL of DCM, and at 0° C., 50 mL of TFA was added. The solution was stirred for reaction and in the end the solvent was removed to yield an oily product (H-Gly-Gly-OBzl.TFA).

5. Preparation of Z-Gly-Gly-Gly-Gly-OBzl

36 g of H-Gly-Gly-OBzl.TFA was dissolved in 500 mL of DMF, and at 0° C. NMM was added to adjust the pH value of the solution to 7. To the solution, Z-Gly-Gly-OH (22.64 g), HOBt (12.16 g), and EDC.HCl (17.25 g) were further added, and the pH value thereof was adjusted with NMM to 8. The cold bath was removed so that the chemical reactions took place at room temperature. After the reaction, a solid precipitate was produced. The precipitate was filtered, collected, washed separately with 5% H₃PO₄ and saturated NaHCO₃ solution, and dried to yield a solid (Z-Gly-Gly-Gly-Gly-OBzl).

6. Preparation of H-Gly-Gly-Gly-Gly-OH

38.5 g of Z-Gly-Gly-Gly-Gly-OBzl was dissolved in 1 L of DMF at 40° C., Pd/C (10%, 10 g) added. The solution was carried out with a hydrogenation reaction. Subsequently, the solution was filtered and the filter residue collected. The residue was stirred with 200 mL of 1N NaHCO₃. The resulting mixture was filtered and the filtrate was collected for next step.

7. Preparation of Fmoc-Gly-Gly-Gly-Gly-OH

The filtrate collected from the step 6) was cooled using a cold bath and THF solution containing Fmoc-OSu was added. The cold bath was removed so that the chemical reactions took place at room temperature. 1N HCl was added to adjust the pH value to between 2 and 3. A precipitate was filtered, collected, washed with water and EA, and dried to yield a solid.

The solid was dissolved in DMF at 70° C. The solution was added to a mixture comprising water and saturated salt water, each of which has a volume four times as much as that of the solution. A precipitate was produced, which was washed with water and dried to yield a product (Fmoc-Gly-Gly-Gly-Gly-OH, with a purity of 98.54%).

### B. Preparation of Fmoc-Asn(Trt)-Gly-OBzl Using Liquid Phase Synthesis Method

The reaction formula for the preparation of Fmoc-Asn(Trt)-Gly-OBzl is summarized as below:

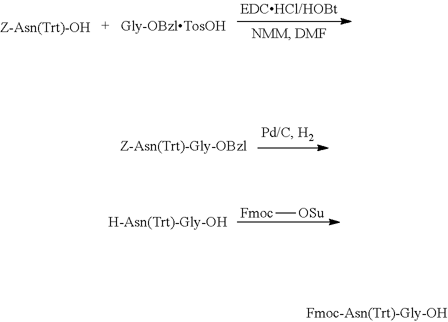

1. Preparation of Z-Asn(Trt)-Gly-OBzl

10.17 g of Z-Asn(Trt)-OH, 7.42 g of H-Gly-OBzl.TosOH, and 2.97 g of HOBt were dissolved in DMF. The solution was cooled to 0° C. using a cold bath and then 4.22 g of EDC.HCl were added. The pH value of the solution was adjusted to 8 with NMM and then the cold bath was removed so that the chemical reactions took place at room temperature. After the reaction, the solution was diluted with EtOAc (EA), washed with 5% H₃PO₄, and extracted with EA. The organic phase was combined and washed separately with 5% H₃PO₄ (80 mL) thrice and saturated salt water once. A precipitate was collected, filtered, dried, and concentrated to yield a crude product which was re-crystallized with 50 mL of EA to yield a white solid (Z-Asn(Trt)-Gly-OBzl, with a purity of 99.78%).

2. Preparation of Fmoc-Asn(Trt)-Gly-OH

3.66 g of Z-Asn(Trt)-Gly-OBzl was dissolved in 35 mL of DMF, and Pd/C added. The solution was carried out with a hydrogenation reaction. Subsequently, the solution was diluted with 40 mL of water, stirred for 10 min, and filtered. The filtrate was collected and cooled to 0° C. and then 1.88 g of Fmoc-OSu added. The pH value of the solution was adjusted to 8 using 1N NaOH and then to 8-9 using 10% Na₂CO₃. When the solution was turbid, it was cooled using an ice bath, and the pH value thereof was adjusted to 2-3. The resulting solid was placed in a refrigerator at 2-8° C. overnight, filtered, washed with water, and dried to yield a crude product with a purity of 75.90%.

The crude product was heated and dissolved in 500 mL of MeOH, and then cooled to room temperature and placed in a refrigerator. The solution was filtered and dried to yield a product with a purity of 98.7%.

### C. Preparation of D-Phe-Pro-Arg-Pro-Gly-Gly-Gly-Gly-Asn-Gly-Asp-Phe-Glu-Glu-Ile-Pro-Glu-Glu-Tyr-Leu Using Solid Phase Synthesis Method

Loading: An amino acid resin was put into a solid-phase synthesis reactor. Each gram of the resin was swollen with 10-15 mL of DMF for 2-3 hrs.

Bonding of Fmoc- Leu: 2.0 molar equivalents of Fmoc-Leu-OH was activated with 2,6-dichlorobenzoylchloride and pyridine and reacted with Wang resin (having a substitution rate of 0.6-1.4 mmol/g) in a DMF solution.

Removing Fmoc: Another DMF solution comprising 15% of piperidine/5% of DBU was added and allowed to react for 30 min so as to remove Fmoc. The resultant resin was washed once with DMF, thrice with methanol, and thrice with DMF, respectively.

Condensing Fmoc-Gly-Gly-Gly-Gly-OH: 3.0 resin equivalent of Fmoc-Gly-Gly-Gly-Gly-OH and 3.0 resin equivalent of HOBt were dissolved with DMF (3 mL/g resin); the mixture was added to the resin, and then 3.0 resin equivalent of DIC was added. The whole process was monitored by ninhydrin colorimetric method (Kaiser). The resin was washed with methanol and DMF.

Condensing Fmoc-amino acid (the last was Boc-D-Phe-OH): 3.0 resin equivalent of Boc-D-Phe-OH (the No. 1 amino acid) and 3.0 resin equivalent of HOBt were dissolved with DMF (3 mL/g resin); the mixture was added to the resin, and then 3.0 resin equivalent of DIC was added.

Condensing Fmoc-Arg(Pbf)-OH: 3.0 equivalents of Fmoc-Arg(Pbf)-OH and pentafluorophenol were dissolved with DMF (3 mL/g resin), and then 3.0 equivalents of DIC were added and stirred for 45 min. The resultant Fmoc-Arg(Pbf)-OPfp/DMF solution was added to the resin and stirred for 3 hrs. If the condensation reaction was not complete in 21-24 hrs, repeat the above steps.

Monitoring the condensation: The whole process was monitored by ninhydrin colorimetric method (Kaiser). The peptide condensation after the introduction of proline (Pro) was monitored using Chloranil and Kaiser test method.

Washing: After all required amino acids were condensed, the resin was washed with methanol and DMF and dried to a constant weight, and a yield thereof was calculated according to its weight gain.

Preparation of a cleavage agent: TFA, TIS, and water with a volume ratio of 95:2.5:2.5 (±10%) were mixed in a vessel to yield a cleavage agent.

Cleavage: The peptide resin was slowly added to a cooled cleavage agent. The mixture was held at 0-15° C. and then increases to 18-28° C., and stirred for 2-3 hrs. The mixture was filtered and the resin washed with TFA (0.5-10. mL TFA/g resin). The resultant filtrate was collected.

Precipitating: To MTBE cooled to −8° C., the filtrate was added and a peptide precipitate was produced. The precipitate stood for 60-80 min at 0-10° C. and was separated using centrifugation. The collected precipitate was washed with methyl tert-butyl ether and separated again using centrifugation.

Drying: The solid peptide was transferred to a vessel and dried to yield bivalirudin II.

Analysis showed that the impurity Bivalirudin−Gly was 0.19% in content, and the impurity Bivalirudin+Gly was 0.42% in content.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A method for producing bivalirudin using solid phase peptide synthesis, comprising:
a) in the presence of a condensing agent, condensing Fmoc-Asn(Trt)-Gly-OH with a peptide resin represented by Formula I (SEQ ID NO. 1);
Asp(OtBu)¹¹-Phe¹²-Glu(OtBu)¹³-Glu(OtBu)¹⁴-Ile¹⁵-Pro¹⁶-Glu(OtBu)¹⁷-Glu(OtBu)¹⁸-Tyr(tBu)¹⁹-Leu²⁰-Resin (I)
to yield a peptide resin represented by Formula II (SEQ ID NO. 2);
Fmoc-Asn(Trt)⁹-Gly¹⁰-Asp(OtBu)¹¹-Phe¹²-Glu(OtBu)¹³-Glu(OtBu)¹⁴-Ile¹⁵-Pro¹⁶-Glu(OtBu)¹⁷-Glu(OtBu)¹⁸-Tyr(tBu)¹⁹-Leu²⁰-Resin (II)
b) mixing the peptide resin represented by Formula II with a de-protective agent to remove Fmoc- and yield a peptide resin represented by Formula III (SEQ ID NO. 3);
Asn(Trt)⁹-Gly¹⁰-Asp(OtBu)¹¹-Phe¹²-Glu(OtBu)¹³-Glu(OtBu)¹⁴-Ile¹⁵-Pro¹⁶-Glu(OtBu)¹⁷-Glu(OtBu)¹⁸-Tyr(tBu)¹⁹-Leu²⁰-Resin (III)
c) in the presence of the condensing agent, condensing Fmoc-Gly-Gly-Gly-Gly-OH with the peptide resin represented by Formula III to yield a peptide resin represented by Formula IV (SEQ ID NO. 4);
Fmoc-Gly⁵-Gly⁶-Gly⁷-Gly⁸-Asn(Trt)⁹-Gly¹⁰-Asp(OtBu)¹¹-Phe¹²-Glu(OtBu)¹³-Glu(OtBu)¹⁴-Ile¹⁵-Pro¹⁶-Glu(OtBu)¹⁷-Glu(OtBu)¹⁸-Tyr(tBu)¹⁹-Leu²⁰-Resin (IV)
d) separately condensing Pro, Arg, Pro, and Phe with the peptide resin represented by Formula IV from C-terminal to N-terminal to yield a peptide resin represented by Formula V (SEQ ID NO. 5),
Boc-D-Phe¹-Pro²-Arg(Pbf)³-Pro⁴-Gly⁵-Gly⁶-Gly⁷-Gly⁸-Asn(Trt)⁹-Gly¹⁰-Asp(OtBu)¹¹-Phe¹²-Glu(OtBu)¹³-Glu(OtBu)¹⁴-Ile¹⁵-Pro¹⁶-Glu(OtBu)¹⁷-Glu(OtBu)¹⁸-Tyr(tBu)¹⁹-Leu²⁰-Resin (V)
e) in the presence of a cleavage agent, separating a peptide from the peptide resin represented by Formula V to yield bivalirudin represented by Formula VI (SEQ ID NO. 6).
D-Phe-Pro-Arg-Pro-Gly-Gly-Gly-Gly-Asn-Gly-Asp-Phe-Glu-Glu-Ile-Pro-Glu-Glu-Tyr-Leu (VI)

2. The method of claim 1, wherein Fmoc-Asn(Trt)-Gly-OH is synthesized as follows: a) mixing Z-Asn(Trt)-OH with H-Gly-OBzl.TosOH so that a liquid phase peptide condensation reaction happens between the two to yield Z-Asn(Trt)-Gly-OBzl; b) reducing Z-Asn(Trt)-Gly-OBzl with hydrogen to yield H-Asn(Trt)-Gly-OH; and c) mixing H-Asn(Trt)-Gly-OH with Fmoc to yield Fmoc-Asn(Trt)-Gly-OH.

3. The method of claim 2, wherein Fmoc-Gly-Gly-Gly-Gly-OH is synthesized as follows: a) mixing H-Gly-Gly-OBzl with Z-Gly-Gly-OH so that a liquid phase peptide condensation reaction happens between the two to yield Z-Gly-Gly-Gly-Gly-OBzl; b) reducing Z-Gly-Gly-Gly-Gly-OBzl with hydrogen to yield H-Gly-Gly-Gly-Gly-OH; and c) mixing H-Gly-Gly-Gly-Gly-OH with Fmoc to yield Fmoc-Gly-Gly-Gly-Gly-OH.

4. The method of claim 3, wherein H-Gly-Gly-OBzl is synthesized by condensing Boc-Gly-OH and H-Gly-OBzl using liquid phase peptide condensation and then removing protecting groups of the condensate.

5. The method of claim 3, wherein Z-Gly-Gly-OH is synthesized by condensing Z-Gly-OH and H-Gly-OMe using liquid phase peptide condensation and then reducing the condensate.

6. The method of claim 1, wherein based on its total volume, the de-protective agent comprises between 3 and 20% of piperidine and between 0.5 and 10% of bicyclic amidine.

7. The method of claim 6, wherein the de-protective agent further comprises between 0 and 20% of 1-hydroxy benzotriazole, between 0 and 8% of 3-hydroxy-1,2,3-benzo triazine-4(3H)-one, or a mixture thereof.

8. The method of claim 1, wherein in the step d), upon condensing Arg, Fmoc-Arg(Pbf)-OH, pentafluorophenol, and the condensing agent are mixed so as to prompt the condensation of Fmoc-Arg(Pbf)-OH with the peptide bound to the resin.

9. The method of claim 1, wherein the condensing agent is N,N′-diisopropyl carbodiimide, O-(7-aza-benzotriazole-1-yl)-N,N,N′,N′-tetramethyl uronium hexafluoro phosphate, O-(benzotriazole-1-yl)-N,N,N,N-4-methyl-uronium tetrafluoroborate/N-methyl morpholine or diisopropyl ethylamine, O-(7-benzotriazole-1-yl)-N,N,N′,N′-tetramethyl uronium hexafluoro phosphate/N-methyl morpholine or diisopropyl ethylamine, (benzo triazol-1-yl-O)tripyrrolidine phosphonium hexafluorophosphate, 1-hydroxy benzotriazole, or a mixture thereof.

10. The method of claim 1, wherein the peptide condensation process is monitored using ninhydrin colorimetric method.

11. The method of claim 1, wherein the cleavage agent comprises trifluoroacetic acid, triisopropyl silane, and water, with a volume ratio thereof 95-60: 5-10: 5-30.

12. A method for producing bivalirudin using solid phase peptide synthesis, the method comprising
a) in the presence of a condensing agent, condensing Fmoc-Gly-Gly-Gly-Gly-OH with a peptide resin represented by Formula III (SEQ ID NO. 3);
Asn(Trt)⁹-Gly¹⁰-Asp(OtBu)¹¹-Phe¹²-Glu(OtBu)¹³-Glu(OtBu)¹⁴-Ile¹⁵-Pro¹⁶-Glu(OtBu)¹⁷-Glu(OtBu)¹⁸-Tyr(tBu)¹⁹-Leu²⁰-Resin (III)
to yield a peptide resin represented by Formula IV (SEQ ID NO. 4);
Fmoc-Gly⁵-Gly⁶-Gly⁷-Gly⁸-Asn(Trt)⁹-Gly¹⁰-Asp(OtBu)¹¹-Phe¹²-Glu(OtBu)¹³-Glu(OtBu)¹⁴-Ile¹⁵-Pro¹⁶-Glu(OtBu)¹⁷-Glu(OtBu)¹⁸-Tyr(tBu)¹⁹-Leu²⁰-Resin (IV)
b) separately condensing Pro, Arg, Pro, and Phe with the peptide resin represented by Formula IV from C-terminal to N-terminal to yield a peptide resin represented by Formula V (SEQ ID NO. 5);
Boc-D-Phe¹-Pro²-Arg(Pbf)³-Pro⁴-Gly⁵-Gly⁶-Gly⁷-Gly⁸-Asn(Trt)⁹-Gly¹⁰-Asp(OtBu)¹¹-Phe¹²-Glu(OtBu)¹³-Glu(OtBu)¹⁴-Ile¹⁵-Pro¹⁶-Glu(OtBu)¹⁷-Glu(OtBu)¹⁸-Tyr(tBu)¹⁹-Leu²⁰-Resin (V)
and
c) in the presence of a cleavage agent, separating a peptide from the peptide resin represented by Formula V to yield bivalirudin represented by Formula VI (SEQ ID NO. 6).
D-Phe-Pro-Arg-Pro-Gly-Gly-Gly-Gly-Asn-Gly-Asp-Phe-Glu-Glu-Ile-Pro-Glu-Glu-Tyr-Leu (VI)

13. The method of claim 12, wherein based on its total volume, the de-protective agent comprises between 3 and 20% of piperidine and between 0.5 and 10% of bicyclic amidine.

14. The method of claim 13, wherein the de-protective agent further comprises between 0 and 20% of 1-hydroxy benzotriazole, between 0 and 8% of 3-hydroxy-1,2,3-benzo triazine-4(3H)-one, or a mixture thereof.

15. The method of claim 12, wherein the cleavage agent comprises trifluoroacetic acid, triisopropyl silane, and water, with a volume ratio thereof 95-60:5-10:5-30.


SEQUENCE LISTING (6 sequences)

SEQ ID NO: 1 - protein, 10 aa, Artificial
  Fully synthetic peptide
DFEEIPEEYL

SEQ ID NO: 2 - protein, 12 aa, Artificial
  Fully synthetic peptide
NGDFEEIPEEYL

SEQ ID NO: 3 - protein, 12 aa, Artificial
  Fully synthetic peptide
NGDFEEIPEEYL

SEQ ID NO: 4 - protein, 16 aa, Artificial
  Fully synthetic peptide
GGGGNGDFEEIPEEYL

SEQ ID NO: 5 - protein, 20 aa, Artificial
  Fully synthetic peptide
FPRPGGGGNGDFEEIPEEYL

SEQ ID NO: 6 - protein, 20 aa, Artificial
  Fully synthetic peptide
FPRPGGGGNGDFEEIPEEYL